(19) United States
(12) Patent Application Publication
(10) Pub. No.: US 2014/0275299 A1
(43) Pub. Date: Sep. 18, 2014

(54) Systems and Methods for Hydrothermal Conversion of Biomass

(71) Applicant: Algae Systems, LLC, Carson City, NV (US)

(72) Inventors: William Brian Bedwell, Palo Alto, CA (US); Matthew Atwood, Chicago, IL (US); Jin-Ping Lim, Palo Alto, CA (US); Esperanza Alvarez, Palo Alto, CA (US); Jordi Perez Mariano, Palo Alto, CA (US)

(21) Appl. No.: 13/829,098

(22) Filed: Mar. 14, 2013

(51) Int. Cl. C10L 1/18; C07C 1/12; C01B 3/34

(52) U.S. Cl. CPC C10L 1/1802; C01B 3/34; C07C 1/12
USPC 518/704; 44/300; 435/167; 252/373

(57) ABSTRACT

Systems and methods are provided for synthesizing low nitrogen concentration organic liquids from biomass, such as algae, the organic liquids being suitable for refining into fuels and other chemicals. The biomass together with a solvent that is immiscible with water at room temperature are subjected to a subcritical hydrothermal treatment to disrupt cell structure and transform the biomass into a gas phase, a bio-oil phase, an aqueous phase and a solid phase. The aqueous phase contains most of the nitrogen. The resulting multi-phasic mixture can be separated into four phases, including an aqueous phase and the organic liquid which consists of bio-oil dissolved in the solvent. Refined organic liquid can be recycled into the hydrothermal treatment as the solvent. The subcritical hydrothermal treatment can be performed at a generally low temperature and followed by a second hydrothermal treatment at a higher temperature.

### CROSS-REFERENCE TO RELATED APPLICATIONS

This application is related to U.S. Non-Provisional patent application Ser. No. 13/______ filed on even date herewith and entitled “Dewatering Systems and Methods for Biomass Concentration” (attorney docket number 5658.16-1 (SMC)) which claims the benefit of U.S. Provisional Patent Application No. 61/664,532 filed on Jun. 26, 2012 and entitled “Dewatering Systems and Methods for Algae Concentration” both of which are incorporated by reference herein.

### BACKGROUND OF THE INVENTION

1. Field of the Invention

The invention relates generally to the field of renewable energy and more particularly to systems and methods for the production of fuels from algae.

2. Description of the Prior Art

Algae are excellent choices as biomass for the production of biofuels because, among other properties, algae can grow very fast and in areas not well suited for other uses like on bodies of water and on non-arable land. Algal biomass is grown in water, and therefore requires a significant amount of energy to fully dry. Hydrothermal liquefaction (HTL) is a convenient thermo-chemical pathway for the production of algae-derived bio-oils because hydrothermal liquefaction does not require the biomass to be fully dried. However, due to the high protein content of algae, the resulting bio-oils generated by hydrothermal liquefaction are rich in nitrogen, often incorporated into aromatic compounds. Such bio-oils are unsuitable for refining by catalytic deoxygenation processes, those typically employed to produce transportation fuels from petroleum-derived oils, because this type of nitrogen is difficult to remove and may poison the catalysts. The production of algal biofuels by hydrothermal liquefaction also is lacking in carbon efficiency in that some valuable carbon-containing molecules cannot be efficiently removed from the aqueous phase, and are lost, unless they are processed by a different route.

Savage et al. (USPGP 2012/0055077) discloses a two-step hydrothermal liquefaction process for converting algae to bio-oil. In the process of Savage et al. a first hydrothermal treatment is performed under subcritical conditions while the second hydrothermal treatment is performed under supercritical conditions.

Gupta et al. (USPGP 2011/0179703) discloses a process for subcritical hydrothermal treatment of biomass to form biochar. Bio-oil is a by-product of the process, and since the bio-oil is not considered desirable, but merely wasted carbon, the bio-oil is recycled back into the subcritical hydrothermal treatment, serving to boost the biochar yield. While the process of Gupta et al. can be used with algae as the biomass, Gupta et al. does not describe using algae and instead lists examples of biomass that yield more significant amounts of biochar such as “forestry or agricultural waste products, wood logs, wood slabs, wood chips, bark, corn-based products, wheat straw, nutshells, [and] sugar cane.” These sources are generally high in lignin, unlike algae.

### SUMMARY

An exemplary process of the present invention comprises a first hydrothermal liquefaction treatment followed by a separation step. In the first hydrothermal treatment an aqueous suspension of a biomass, such as algae, and a first organic solvent are treated together under a first subcritical hydrothermal condition comprising a first treatment temperature of between about 150° C. to about 350° C. and a pressure of between about 500 psi to about 3000 psi to produce a first multi-phasic mixture including a gas phase, an organic liquid phase, an aqueous liquid phase and a solid phase. The organic solvent is characterized by being immiscible with water at room temperature and can comprise, for example, a hydrocarbon. In various embodiments the aqueous suspension of the biomass includes between about 5% to about 50% of the biomass by weight. In various embodiments the volume ratio of water to the first organic solvent used in the subcritical treatment is between about 1:1 to about 10:1. Optionally, additives such as a catalyst can be added to the first hydrothermal treatment.

The separation step serves to separate the organic phase from the aqueous phase of the multi-phasic mixture and optionally also produces a gaseous phase and/or a solid phase. In various embodiments the exemplary process comprises recovering carbon from the aqueous phase produced by the separation step. In some embodiments the gaseous phase is processed along with the aqueous phase. Recovered carbon, as carbon dioxide, can be recycled back to algae cultivation, can be converted to liquid hydrocarbon products, can be converted to methane, can be captured and concentrated, or can be recycled back into the hydrothermal treatment, for example. In various embodiments the aqueous phase is provided to a digester to generate biogas, and in these embodiments the biogas can then be subjected to steam methane reforming to convert the methane fraction thereof to hydrogen and carbon dioxide, or the biogas can be subjected to steam gasification to convert the methane fraction thereof to syngas. In these latter embodiments, the syngas is optionally further processed to produce a liquid hydrocarbon.

The exemplary process can also include steps both before the first hydrothermal treatment and after the first separation step. Thus, in some embodiments the process also comprises producing the aqueous suspension of the biomass before the first hydrothermal treatment where producing the aqueous suspension of the biomass includes cultivating algae and dewatering the algae. In further embodiments, the process further comprises converting the organic phase to a fuel, and in some of these embodiments the process further comprises feeding some of the fuel back into the first hydrothermal treatment as at least a portion of the first organic solvent.

In still further embodiments of the exemplary process the first temperature is no more than about 200° C. In some of these embodiments the first multi-phasic mixture further includes a solid phase and the separation step further comprises separating the solid phase from the organic and aqueous phases. In these embodiments the method also further comprises a second hydrothermal treatment comprising treating the solid phase under a second hydrothermal condition, optionally together with a second solvent, where the second hydrothermal condition comprises a second treatment temperature above the first treatment temperature and a pressure of about 200 psi to about 3000 psi to produce a second multi-phasic mixture of an organic phase and an aqueous phase, and separating the organic phase from the aqueous phase of the second multi-phasic mixture. The second hydrothermal condition is optionally either subcritical or supercritical. In further of these embodiments the process additionally comprises converting the organic phases recovered from separating the first and second multi-phasic mixtures to a fuel. Optionally, the process can additionally comprise recovering carbon and nutrients from the aqueous phase separated from the second multi-phasic mixture by the methods already noted.

The present invention also provides products by the processes disclosed herein. In particular, products produced by these processes when the biomass comprises algae or microalgae include low nitrogen concentration organic phases suitable for refining.

### BRIEF DESCRIPTION OF DRAWINGS

FIG. 1 is a flowchart representation of a method according to an exemplary embodiment of the present invention.

FIG. 2 is a flowchart representation of a method according to another exemplary embodiment of the present invention.

### DETAILED DESCRIPTION OF THE INVENTION

The present invention provides systems and methods for synthesizing a low nitrogen concentration organic product from biomass, the organic product being suitable for refining into hydrocarbons such as transportation fuels. The methods subject the biomass together with a solvent to a subcritical hydrothermal treatment that separates the biomass into several phases including an organic phase and an aqueous phase. The organic phase, the product of the process, includes bio-oil, also known as bio-crude oil, derived from lipids in the biomass, while the aqueous phase includes a water solution of the hydrolysis products of biomolecules like proteins and carbohydrates from the biomass, and the water-soluble molecules formed by reactions of these products. The solids are comprised of insoluble proteins and carbohydrates. The solvent serves to extract non-polar molecules while the aqueous phase dissolves nitrogen-containing molecules, leaving the organic phase, comprising the bio-oil dissolved in the solvent, with low nitrogen. Further embodiments employ two stages, a first subcritical hydrothermal treatment followed by a first separation step in the first stage followed by a second hydrothermal treatment and a second separation step, where the temperature of the second hydrothermal treatment is greater than the first. In these two-stage processes a solid phase recovered from the first separation is treated in the second hydrothermal treatment, which optionally can be either subcritical or supercritical. If the first step is done in mild conditions, the nitrogen-containing molecules in the aqueous phase do not react to form larger polymers that could get incorporated into the organic phase. Rather, the nitrogen-containing molecules stay in the aqueous phase.

FIG. 1 serves to illustrate various exemplary embodiments of the invention. An illustrated process 100 begins with an optional step 110 of obtaining a biomass. Next, the biomass together with a solvent are subjected to a subcritical hydrothermal liquefaction treatment in a step 120 to yield a multi-phasic mixture. A phase separation is performed in a step 130 to separate an organic phase from an aqueous phase and optionally separate out solid and gaseous phases. The organic phase optionally is refined in a step 140. The aqueous phase is optionally further processed in a step 160. Each of these steps is further detailed below. Various steps of the process 100 optionally can be performed in batches or in continuous operation.

The step 110 comprises obtaining a biomass. This step can be performed, for example, by cultivating a biomass such as algae or switchgrass, or by recovering waste products such as agricultural waste, like corn stalks, or waste from the lumber and paper industries. The production of the biomass takes up carbon which is then converted by the process 100 into a useful product. The step 110 is optional in that some processes of the present invention can be practiced without performing a cultivation, waste recovery, or similar step, and in these embodiments the process begins with received biomass. In various embodiments, the biomass has a C:N ratio of less than about 10, or in the range of between 10 and 20, or above 20, while the organic phase produced from the biomass can have a C:N ratio of greater than about 40.

In various embodiments, obtaining the biomass in step 110 can comprise cultivating algae followed by harvesting and dewatering the algae. One advantage of the present invention is that biomass does not have to be excessively dewatered since the subcritical hydrothermal treatment 120 requires water. In the case where the biomass comprises algae, the dewatering step produces an aqueous suspension, or slurry, of algae in water. Dewatering of algae can be performed by such techniques as centrifugation, filtration, and evaporation. “Dewatering Systems and Methods for Algae Concentration,” noted above, provides still further systems and processes for algae dewatering.

Suitable ranges for the concentration of algae in the aqueous suspension include between about 5% to about 30% of the algae by weight, between about 10% to about 50% of the algae by weight, and between about 25% to about 30% of the algae by weight. Other forms of biomass may also have to be dewatered, or have water added, to create an aqueous suspension of the biomass within these ranges. Examples of suitable algae include microalgae, macro algae, blue-green algae, heterotrophic algae, mixotropic algae, cyanobacteria and so forth. Microalgae can be a particularly low-nitrogen form of algae. Biomass characterized by low protein concentration and/or a high lipid concentration are favored for higher bio-oil yields. Algae is generally characterized by a protein concentration of between about 6% to about 50% or more. Other biomass with lower protein concentrations can also be used. Likewise, lipid concentrations in algae can generally be between about 5% to about 45% or even higher for algae grown heterotrophically or mixotrophically; typically lower for other biomass types.

In step 120 the aqueous biomass suspension, together with a solvent 125, is subjected to a subcritical hydrothermal liquefaction treatment in a pressurized vessel to produce a multi-phasic mixture of at least an organic phase and an aqueous phase. More specifically, the solvent 125 comprises an organic solvent 125 characterized by being immiscible with water at room temperature. Examples of suitable organic solvents 125 include long-chain hydrocarbon molecules such as heptane, octane, nonane, decane, dodecane and the like, as well as some medium and short-chain hydrocarbons. Other suitable organic solvents 125 include hydrocarbon mixtures such as gasoline, diesel, and kerosene, aromatic and aliphatic solvents, and hydrogen donor solvents like tetralin. Suitable organic solvents 125 can be non-polar or polar, an example of the latter being long-chain alcohols. It should be noted that a dense gas like CO₂, methane, ethane, propane, butane, etc. under the hydrothermal conditions employed herein can be a suitable substitute for the organic solvent 125 in the processes described herein. A suitable volume ratio of water to organic solvent 125 used in the subcritical treating step 120 is between about 1:1 to about 10:1 with 2:1 being a particular example.

The subcritical hydrothermal treatment of step 120 takes place under conditions of temperature and pressure that are below the critical point of water (commonly referred as subcritical conditions). Under these conditions, several processes take place, such as cell lysis, hydrolysis of biomolecules, reactions between molecules liberated during hydrolysis, and ultimately the formation of bio-oil by polymerization reactions. Generally, lipids and short organic polymers in the biomass become the bio-oil while the proteins and carbohydrate hydrolysis products are water-soluble and are dissolved into the aqueous phase. Proteins and carbohydrates may also form a solid phase, discussed below. The organic solvent 125 is believed to increase the extraction efficiency of the lipids and short organic polymers to generate a higher bio-oil yield. The organic solvent 125 also serves to dissolve the bio-oil such that the organic phase produced in the step 120 is bio-oil dissolved in the organic solvent 125.

The subcritical hydrothermal treatment of step 120 can comprise any combination of temperature and pressure that is below the critical point of water (about 374° C. and 3206 psia), however, in some embodiments the conditions comprise a treatment temperature of between about 150° C. to about 350° C. and a pressure of between about 500 psi to about 3000 psi. In some further embodiments, described in greater detail below, the treatment temperature is no greater than about 200° C. When the subcritical hydrothermal treatment step 120 is performed in a batch mode, the treatment can be carried out in an autoclave, for instance. A high pressure continuous reactor can be employed for continuous operation.

Various embodiments of the process 100 comprise adding additional components, such as a catalyst, to the biomass/solvent combination in step 120. For example, carbonate compounds such as K₂CO₃ are known to aid in the hydrolysis of biomolecules. See, for example, Zhang, Y., Biofuels from Agricultural Wastes and Byproducts, chapter 10, (2010), incorporated herein by reference.

The multi-phasic mixture resulting from the subcritical hydrothermal treatment step 120 is next subjected to a separation step 130 in which the organic phase is separated from the aqueous phase. Solid and gaseous phases can optionally also be separated from the multi-phasic mixture. Examples of separation technologies that can be employed in separation step 130 include separation tanks, decanters, centrifuges, and filtration. In those embodiments in which the subcritical hydrothermal treatment step 120 is performed in a batch mode, the separation step 130 also can be performed in a batch mode, and in the same vessel as was used for the subcritical hydrothermal treatment step 120. In some embodiments, some or all of the gaseous phase is released by the reactor employed in the subcritical hydrothermal treatment step 120. These gases can contain carbonaceous gases such as carbon monoxide and methane, or other lightweight hydrocarbons. Therefore it will be appreciated that the separation step 130 can begin during the subcritical hydrothermal treatment step 120, in some embodiments. Gases released during the subcritical hydrothermal treatment step 120 can optionally be mixed with gases later released while separating the aqueous phase from the organic phase.

The organic phase recovered from the separation step 130 is next optionally refined in a step 140 to produce a product 150. Refining in step 140 can be performed by catalytic hydrogenation/deoxygenation processes commonly used to refine oils produced from petroleum. The product 150 of refining in step 140 can therefore be low-nitrogen transportation fuels such as diesel and gasoline. Other hydrocarbon products 150 can also be produced by such refining. In some embodiments, some of the product 150 of the refining step 140 is used as the solvent 125 in the subcritical hydrothermal treatment step 120. Exemplary products 150 include dodecane, naphtha-like products, gasoline fractions, diesel-like products, and kerosene like products.

As noted above, the aqueous phase produced by the separation step 130 can include soluble carbon-containing molecules that did not end up in the organic phase. In a step 160 the aqueous phase can be further processed to recover such carbon. Step 160 can comprise, in various embodiments, steam reforming, steam gasification, hydroprocessing, direct hydrotreatment, aerobic and anaerobic digestion, as well as aqueous chemical processing, such as by precipitation. These processes can either produce carbon dioxide that can be used to cultivate biomass in step 110 as illustrated, or liquid hydrocarbons, or can produce liquid or solid carbon-containing compounds that can be fed directly back into the subcritical hydrothermal treatment step 120 or fed forward into a subsequent hydrothermal treatment, discussed below. Some or all of any carbon oxide gases produced in step 160 can optionally be fed into the subcritical hydrothermal treatment step 120.

In addition to carbon recovery, other nutrients such as nitrogen and phosphorous and other heteroatoms can be recovered in step 160, through some of the noted recovery techniques, to be returned to cultivate the biomass in step 110. Step 160 can comprise, in some instances, also recovering carbon-containing solids and/or gases from the separation step 130, and in some of these embodiments the recovered solids and/or gases are fed into the same process as the recovered aqueous phase. Another product of step 160, in some embodiments, is methane or biogas which can be used as a fuel for heating in steps such as step 120. Hydrogen can be another product of step 160, and the hydrogen can be used to generate electric power, for example or co-fed in the first or second hydrothermal steps 120, 210 (see FIG. 2, below) to maintain a reducing environment, or used to hydrotreat or hydroprocess the resultant organic phase into various fuels.

In an exemplary embodiment, the aqueous phase can be first used to produce biogas, such as by digestion. The biogas from digestion can then be processed by steam methane reforming to convert the methane fraction of the biogas to hydrogen and carbon dioxide, or the biogas can be processed by steam gasification to convert the methane fraction of the biogas to syngas, a gaseous mixture of hydrogen and primarily carbon monoxide sometimes with some carbon dioxide. Syngas can optionally be produced directly from the aqueous phase by steam gasification. Syngas can be used in a Fischer-Tropsch process to produce liquid hydrocarbons. Hydrogen can be recovered from either gas mixture, such as through pressure swing absorption, and the remaining carbon oxide can be fed back into the subcritical hydrothermal treatment step 120, or used for cultivation in step 110 in the case where the carbon oxide is carbon dioxide and the hydrogen gas can be used for hydrotreatment of bio-oil or co-fed in the hydrothermal reactions. In other embodiments the syngas can be combusted directly for heat and power, etc. Solids recovered in separation step 130 optionally also can be subjected to steam gasification separately, or together with biogas, or together with the aqueous phase, to generate syngas.

As previously noted, in some embodiments the temperature of the subcritical hydrothermal treatment step 120 is no more than 200° C. In some of these embodiments the subcritical hydrothermal treatment step 120 is a first such hydrothermal step carried out at a relatively low temperature and in these embodiments a second hydrothermal treatment step and separation step are added to recover carbon from solids produced in the first subcritical hydrothermal treatment step 120.

FIG. 2 serves to illustrate various additional exemplary embodiments of the invention that employ two hydrothermal treatment steps. An illustrated process 200 begins with an optional step 110 of obtaining a biomass as described above. The process 200 continues with a subcritical hydrothermal treatment step 120 as described above, but limited such that the temperature is no more than 200° C. The multi-phasic mixture resulting from the subcritical hydrothermal treatment step 120 is next subjected to a separation step 130, as previously described, in which the organic phase is separated from the aqueous phase. A solid phase is also separated in these embodiments, as well as an optional gas phase.

In a step 210 the solid phase is treated under a second hydrothermal condition that can be subcritical or supercritical, in different embodiments. The second hydrothermal condition comprises a second treatment temperature above the first treatment temperature and a pressure of about 200 psi to about 3000 psi to produce a second multi-phasic mixture of an organic phase and an aqueous phase. In some of these embodiments a second organic solvent 220 is added to the hydrothermal process of step 210. The second organic solvent 220 is also characterized by being immiscible with water at room temperature and can be the same organic solvent 125 used in step 120, or can be a different organic solvent. As in step 120, a catalyst can also be added to the second hydrothermal step 210. In those embodiments in which the second hydrothermal condition is supercritical, the temperature can be in a range of about 380° C. to about 600° C., or about 390° C. to about 500° C., or about 395° C. to about 450° C.

Next, in a step 230, the organic phase of the second multi-phasic mixture is separated from the aqueous phase as described with respect to the separation step 130. The organic phase from the second multi-phasic mixture is next refined in step 140, as described above. In various embodiments the organic phase from the second multi-phasic mixture is mixed with the organic phase from the first multi-phasic mixture and the combination is refined in step 140. Similarly, the aqueous phase from the second multi-phasic mixture is next optionally processed in the aqueous processing step 160, in some embodiments together with the aqueous phase separated from the second multi-phasic mixture in step 130.

FIGS. 1 and 2 also serve to represent exemplary embodiments of biomass conversion systems comprising one or more hydrothermal vessels and one or more separation units, where the vessel is suitable for the specified conditions of the processes 100, 200 and made of a material that will not react with the reactants therein under the hydrothermal conditions, and where the separation unit is capable of separating the phases of the resultant multi-phasic mixture. In some embodiments, the steps 120 and 210 can be accomplished in a continuous steady-state reactor of the plug flow type or the Continuous Stirred Tank Reactor (CSTR) type. Various additional systems of the present invention include sub-systems necessary for biomass production, such as photobioreactors for cultivation of algae, and harvesting and dewatering equipment. Other systems of the present invention include equipment necessary for the further processing of recovered aqueous phases from separation steps 130, 230, such as gasifiers and digesters.

The present invention also provides compositions of matter in the form of the products synthesized by the processes described herein. It is noted that the organic phases produced by the subcritical hydrothermal treatments described herein are themselves a mixture of bio-oil and the organic solvent that is employed, making these products distinguishable from the bio-oils produced by others, such as the processes provided by Savage et al., even when both processes are fed the same algae.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

What is claimed is:

1. A method comprising:
a first hydrothermal treatment comprising treating together an aqueous suspension of a biomass and a first organic solvent, the first organic solvent being immiscible with water at room temperature, under a first subcritical hydrothermal condition comprising a first treatment temperature of between about 150° C. to about 350° C. and a pressure of between about 500 psi to about 3000 psi to produce a first multi-phasic mixture including an organic phase and an aqueous phase; and
separating the organic phase from the aqueous phase.

2. The method of claim 1 wherein the first organic solvent comprises a hydrocarbon.

3. The method of claim 1 wherein the aqueous suspension of the biomass includes between about 5% to about 30% of the biomass by weight.

4. The method of claim 1 wherein the biomass comprises algae.

5. The method of claim 1 wherein the volume ratio of water to the first organic solvent used in the subcritical treating step is between about 1:1 to about 10:1.

6. The method of claim 1 wherein the biomass has a C:N less than about 10.

7. The method of claim 1 wherein the organic phase, after the separation step, has a C:N of greater than about 40.

8. The method of claim 1 further comprising recovering carbon from the aqueous phase produced by the separation step.

9. The method of claim 8 further comprising providing the aqueous phase produced by the separation step to a digester to generate biogas.

10. The method of claim 9 further comprising steam methane reforming of the biogas to convert the methane fraction thereof to hydrogen and carbon dioxide.

11. The method of claim 9 further comprising steam gasification of the biogas to convert the methane fraction thereof to syngas.

12. The method of claim 11 further comprising processing the syngas to produce a liquid hydrocarbon.

13. The method of claim 8 wherein recovering carbon from the aqueous phase includes producing carbon dioxide.

14. The method of claim 13 wherein the carbon dioxide is used to cultivate algae.

15. The method of claim 13 wherein the carbon dioxide is fed into the first hydrothermal treatment.

16. The method of claim 1 further comprising adding a catalyst to the first hydrothermal treatment.

17. The method of claim 1 further comprising producing the aqueous suspension of the biomass before the first hydrothermal treatment.

18. The method of claim 17 wherein producing the aqueous suspension of the biomass includes cultivating algae and dewatering the algae.

19. The method of claim 1 further comprising converting the organic phase to a fuel.

20. The method of claim 19 further comprising feeding some of the fuel back into the first hydrothermal treatment as at least a portion of the first organic solvent.

21. The method of claim 1 wherein the first temperature is no more than about 200° C.

22. The method of claim 21 wherein
the first multi-phasic mixture further includes a solid phase,
the separation step further comprises separating the solid phase from the organic and aqueous phases, and
the method further comprises
a second hydrothermal treatment comprising treating the solid phase under a second hydrothermal condition comprising a second treatment temperature above the first treatment temperature and a pressure of about 200 psi to about 3000 psi to produce a second multi-phasic mixture of an organic phase and an aqueous phase, and
separating the organic phase from the aqueous phase of the second multi-phasic mixture.

23. The method of claim 22 further comprising converting the organic phases recovered from separating the first and second multi-phasic mixtures to a fuel.

24. The method of claim 22 wherein the second hydrothermal treatment further includes treating the solids together with a second solvent.

25. The method of claim 22 further comprising recovering carbon from the aqueous phase separated from the second multi-phasic mixture.

26. The method of claim 22 wherein the second hydrothermal treatment comprises a second subcritical hydrothermal treatment.

27. The method of claim 22 wherein the second hydrothermal treatment comprises a supercritical hydrothermal treatment.

28. The method of claim 22 wherein carbon recovered from either aqueous phase is fed into the second hydrothermal treatment as carbon dioxide.

29. A product consisting of the organic phase produced by the process of claim 4.

30. The product of claim 29 wherein the algae consists essentially of microalgae.

31. A product consisting of the organic phase produced by the process of claim 22, wherein the biomass comprises algae.

32. The method of claim 1 wherein the first hydrothermal treatment is accomplished in a continuous steady-state reactor.
